(19) United States
(12) Patent Application Publication
(10) Pub. No.: US 2025/0040878 A1
(43) Pub. Date: Feb. 6, 2025

(54) AI-Powered Smart Bassinet with Multi-Sensor Integration and Real-Time Personalized Infant Care Recommendations

(71) Applicant: BABYMAZING SOLUTIONS, LLC, RESTON, VA (US)

(72) Inventors: Daniel Quintino Rocha, Arlington, VA (US); Rafaela Brasil, Arlington, VA (US)

(73) Assignee: BABYMAZING SOLUTIONS, LLC, RESTON, VA (US)

(21) Appl. No.: 18/793,719

(22) Filed: Aug. 2, 2024

Related U.S. Application Data
(60) Provisional application No. 63/517,213, filed on Aug. 2, 2023.

(51) Int. Cl. A61B 5/00; A61G 11/00; G06N 3/098

(52) U.S. Cl. CPC A61B 5/4806; A61B 5/6892; A61B 5/746; A61G 11/00; G06N 3/098; A61B 2503/04; A61B 2560/0247; A61B 2560/0252; A61G 2203/30; A61G 2203/46

(57) ABSTRACT

A sleep system for a baby includes multiple sensors to sense environmental conditions and physiological parameters of the baby. The sensors are located beneath the baby's resting position and lateral to the baby's torso. The system includes a computer processor with a neural network trained to provide caregivers with personalized sleep improvement recommendations. These are delivered via a bi-directional graphical user interface that allows caregivers to provide feedback. The system employs federated learning for enhanced data security and continuous algorithm improvement based on aggregated data from a network of devices. Dual API architecture enables secure on-device data processing and the transmission of encrypted data to third-party developers, fostering an open ecosystem for innovation in infant care.

### RELATED APPLICATION(S)

This application claims priority to U.S. Provisional Application No. 63/517,213 filed Aug. 2, 2023. The disclosure of the prior application is incorporated herein by reference in its entirety.

### BACKGROUND OF THE DISCLOSURE

### 1. Field of the Disclosure

The present disclosure relates to the field of infant care devices. More specifically, it relates to a smart bassinet system that leverages multiple sensors, deep learning methodologies, predictive modeling, and APIs (Application Programing Interfaces) to gather and analyze data about an infant's physiological and environmental state. The bassinet is further distinguished by its innovative use of multi-source learning techniques, including federated learning and crowd-sourced data analysis, as well as IoT (Internet of Things) control capabilities, to draw insights from a broad range of infant data beyond the individual user and enable third-party application development.

### 2. Description of the Related Art

Parental management of an infant's sleep routine poses significant challenges and concerns, with a common goal of establishing a sleep routine that ensures the infant's health, safety, and comfort. Issues such as Sudden Infant Death Syndrome (SIDS), sleep disturbances, and the resultant stress and anxiety experienced by parents underscore the need for more sophisticated solutions. While traditional infant monitoring devices provide some level of insight, they are often limited by their inability to integrate comprehensive data and provide real-time, actionable recommendations.

Recent technological advancements, particularly in AI, machine learning, and sensor technology, offer a transformative opportunity to address these issues. Despite these advancements, existing solutions for infant care fail to utilize the potential of federated learning and crowd-sourcing data from a large user base to enhance the accuracy and effectiveness of recommendations. Federated learning, which allows for decentralized data processing, ensures the privacy and security of sensitive data while still enabling robust analysis across a vast number of devices. Yet, federated learning has yet to be applied to infant care.

Several notable patents exist in this domain. U.S. Pat. No. 4,640,034 (Zisholtz, Feb. 3, 1987) and U.S. Pat. No. 4,777,938 (Sirota, Oct. 18, 1988) describe various infant sleep devices that use sensory stimuli to induce sleep in children. U.S. Pat. No. 7,127,074 (Landa, Oct. 24, 2006) introduces a baby monitor device that assists a caregiver in training a child to sleep by muting the sound of the child for a fixed period of time. However, these devices do not address the root causes of sleep problems in children. Rather, these devices function to mitigate sleep disruption.

U.S. patent application No. 20150094830 A1 (Rest Devices Inc., Apr. 2, 2015) discloses a computerized health and sleep monitor that captures biometric data of an infant and transmits the data via a network to an event server for evaluation. While this system provides a foundation for health monitoring, it fails to leverage federated learning and APIs to significantly enhance data processing and ensure robust privacy.

World Patent Document WO 02017196695 (“'695”) (Udisense Inc., Nov. 16, 2017) describes a video monitoring system with a fixed camera above a crib. The camera is only capable of watching the baby and cannot measure physiological parameters of the baby. Without these parameters, '695 fails to recognize the needs of the baby and therefore, cannot provide recommendations.

U.S. Pat. No. 9,694,156 (Eight Sleep, Jul. 4, 2017) describes a sleep system with sensors and machine learning for sleep improvement, primarily designed for adults and lacking the comprehensive approach needed for infants. It does not utilize federated learning or APIs for third-party application development.

U.S. Published application No. 20170043118 (Happiest Baby Inc., Feb. 16, 2017) discloses a sleep-aid device that moves and generates sound to calm a baby. While effective for calming, our bassinet also gathers extensive physiological and environmental data, processes this data using AI, and provides real-time insights and recommendations. Additionally, U.S. Published application No. 20170055898 (Awardables Inc., Mar. 2, 2017) describes systems for determining sleep stages and sleep events using sensor data. By incorporating a broader range of sensors and utilizing federated learning, our system enhances data privacy and accuracy, offering more comprehensive insights and recommendations.

U.S. Pat. No. 8,562,511 (Koninklijke Philips N.V., Oct. 22, 2013) describes a system for inducing sleep using a breathing rate measuring unit and light pattern generator. Unlike this system, ours not only induces sleep but also monitors and analyzes a wide range of physiological data, offering personalized recommendations and supporting third-party application development. Additionally, U.S. Pat. No. 8,532,737 (Cervantes, Sep. 10, 2013) discloses an apparatus for automatically monitoring sleep with a video recorder and real-time image transmission. Our system integrates multiple types of sensors beyond video, utilizes AI for data analysis, and provides an open API for third-party developers.

U.S. Pat. No. 9,530,080 (Joan and Irwin Jacobs Technion-Cornell Institute, Dec. 27, 2016) discloses systems for monitoring babies with cameras and centralized computation. However, it does not disclose physiological sensors integrated into a bassinet nor does it employ federated learning for privacy-preserving data analysis. Additionally, U.S. Pat. No. 9,572,376 (Nested Bean Inc., Feb. 21, 2017) describes a wearable accessory that provides gentle pressure to mimic a human hold. By integrating sensors into the bassinet, our proposed solution avoids reliance on wearables, offering a more comprehensive monitoring solution

U.S. Published Application No. 2011/0015467 (“'467”) (Dothie et al.) describes a base unit with sensors for sleep-relevant characteristics and environmental conditions. However, '467 does not address how to monitor an infant in a bassinet as well as optimal placement of the sensors for the infant. Additionally, U.S. Pat. No. 9,694,156 (Eight Sleep, Jul. 4, 2017) details a bed device system for gathering and analyzing human biological signals. This system also does not address the optimal sensor placement and sleeping device for an infant.

U.S. patent application No. 20070279234 (Walsh, Dec. 6, 2007) and European Patent 1810710 (Jul. 25, 2007) present sleep improvement systems that leverage individual information to control the sleeping environment and monitor the quality of sleep, respectively. For example, sensors monitor physiological signals such as snoring, breathing, body movement, or body temperature, providing a comprehensive view of the user's sleep state. However, these systems focus primarily on adult sleep patterns and are not fully adaptable for infant care because infants have significantly different physiological patterns compared to adults. The sensitivity and placement of sensors might need adjustment for infants, and safety concerns are paramount, as devices designed for adults may not meet the stringent safety standards required for infants. Additionally, the mechanisms used to control the sleeping environment for adults might not be suitable or necessary for infants, who require different thermal environments and monitoring for additional parameters like oxygen saturation levels to prevent conditions like sudden infant death syndrome (SIDS).

U.S. Published application No. 20160293042 (“'042”) (Smilables Inc., Oct. 6, 2016) discloses mechanisms for monitoring an infant's emotional state using an infant monitoring hub. While '042 emphasizes emotional state monitoring, the system of '042 does not incorporate a wide array of physiological sensors nor does take advantage of federated learning for more comprehensive data analysis and personalized care recommendations.

Moreover, U.S. patent application No. 20070191692 (Hsu, Aug. 16, 2007) outlines a method for using sensor data to select recommendations for behavioral programs or actions to improve sleep behavior. While the approach is comprehensive, it will lack the ability to customize recommendations based on the unique patterns and needs of an individual infant.

World Patent Document WO 2005089649 (Sep. 29, 2005) proposes an implantable medical device to determine a patient's sleep quality. This approach, however, may not be suitable or desired for infants due to the invasiveness of the device.

Therefore, there is a need for an improved infant sleep system and bassinet with that employs advanced sensor technology, including crying recognition, machine learning algorithms, deep learning techniques, and multi-source data for a comprehensive approach to infant sleep and care routines. Further there is a need, for an improved infant sleep system that integrates multiple sensors within a bassinet, employs advanced machine learning algorithms for real-time recommendations, and facilitates third-party development through open APIs, thereby addressing several limitations of the prior art.

### BRIEF SUMMARY OF THE DISCLOSURE

The infant sleep system, according to this disclosure, may be incorporated into infant care devices such as a bassinet, crib or incubator and enables comprehensive monitoring and improved management of an infant's sleep routine, providing real-time, personalized recommendations for infant care. Such recommendations are the result of extensive data analysis of the infant's sleep patterns, environmental and physiological factors, feeding times, diaper changes, mood, and other crucial factors affecting sleep and overall health. This system may identify and understand individual patterns, using a collaborative learning model to offer personalized and proactive recommendations for infant care. Furthermore, the system may include a graphical user interface (“GUI”) to present its findings in an intuitive and user-friendly manner to effectively assist caregivers in implementing optimal care practices.

In addition, the system may incorporate an advanced Electromechanical Film (EMFi) sensor technology that detects changes in pressure, vibration, and deformation, offering a more detailed understanding of the baby's respiration rate, heart rate, movement, and sleep patterns. Furthermore, parent-reported inputs and environmental factors that may not be captured by the bassinet's sensors are integrated into the system's analysis, creating a holistic view of the baby's health and wellness.

By incorporating all these elements, the system provides a unique, holistic, and personalized approach to infant care that not only enhances a caregiver's ability to monitor and manage an infant's sleep routine but also educates them on evidence-based care practices, thereby contributing to the overall wellbeing of the infant.

Further the infant sleep system, according to this disclosure, introduces an intelligent bassinet designed to significantly enhance the way infant care is managed. This bassinet represents a comprehensive integration of various state-of-the-art technologies within a single unit of infant care equipment. The embedded sensors monitor a broad array of parameters, including the infant's body temperature, movement, breathing patterns, heart rate, and sleep schedules, thereby gaining a comprehensive understanding of the infant's unique routine.

Further, the system may include seamless integration of advanced machine learning technologies, federated learning algorithms, and predictive modeling techniques, all contained within a single bassinet. Also, the system multi-source data collected not only from the individual infant using the bassinet but also from a large and growing network of similar smart bassinets forming a user base. This data, processed through federated learning, enables the detection of patterns across the user base while ensuring the privacy and security of individual data.

Also, the system may include a dual-API architecture to enhance functionality and data security. The first API is designed for on-device data processing, where machine learning algorithms directly interact with sensor data to generate real-time insights and recommendations without transmitting raw data externally. This API supports local computations and adaptations, ensuring swift response times and reducing dependency on external data processing.

The second API manages the secure transmission of processed data to external applications and third-party developers. It is crafted to ensure that data, while being accessible for further innovation and application development, remains encrypted and compliant with privacy standards. This second API facilitates the expansion of the ecosystem, allowing third-party developers to create bespoke applications that utilize the insights generated by the bassinet's sensors, thus contributing to an integrated care solution platform.

By analyzing this multi-source data, the bassinet system may generate highly personalized, precise, and age-appropriate recommendations based on developmental milestones for individual infants. These recommendations span from optimal feeding and sleep times to room temperature adjustments and alerts about potential health concerns. Also, these recommendations may be provided to the care giver on the GUI.

Some aspects of this intelligent infant sleep system is its ability to cross-reference data across users, identify patterns among infants with favorable sleep and care routines, and applying these insights to provide evidence-based recommendations to caregivers. Additionally, providing safe data for third-party developers and research institutions brings significant benefits, including fostering innovation, customizability, and rapid development of new applications. This comprehensive approach ensures that the platform not only meets the immediate needs of parents but also contributes to the broader field of infant health and development through data-driven insights and enhanced research capabilities.

Another aspect of the system includes providing real-time feedback and forward-looking predictions, empowering caregivers to adjust their infant care practices promptly based on the insights provided. In essence, this intelligent bassinet presents a data-driven, scientifically backed system that enhances the effectiveness of infant care. The integrated bassinet, with its wealth of sensor data, AI capabilities, and federated learning, forms part of a learning network of bassinets, each contributing to and benefiting from the collective intelligence of the system. A further beneficial aspect of the system includes providing safe data for third-party developers is the potential for rapid innovation and diverse application development, enhancing the overall ecosystem. This not only benefits consumers by providing a wide range of tailored applications and services but also offers valuable data for research institutions. The rich, anonymized dataset can be used to conduct large-scale studies on infant health and development, leading to breakthroughs in pediatric care and early childhood development research. This setup transforms infant care into a more proactive, informed, and effective process, thereby reducing stress and enhancing the quality-of-care practices.

In some aspects, the techniques described herein relate to a sleep system for a baby including: a flat surface configured to support the baby; a physiological sensor positioned beneath the flat surface; a mattress over the flat surface and the physiological sensor such that the physiological sensor is configured to sense from beneath the baby; and at least one lateral sensor connected to the flat surface and configured to sense the baby from a side of the baby, wherein the at least one lateral sensor and the physiological sensor are configured to sense at least one physiological parameter of the baby and generate corresponding sensor data.

In some aspects, the techniques described herein relate to a system, wherein the at least one lateral sensor extends above the mattress.

In some aspects, the techniques described herein relate to a system, further including: a perimeter edge surrounding the flat surface; at least one lateral sensory guard extending vertically from the flat surface, the lateral sensory guard between the perimeter edge and a central portion of the mattress; and the at least one lateral sensor on the lateral sensory guard.

In some aspects, the techniques described herein relate to a system, further including: a perimeter edge surrounding the flat surface; a side wall connected to the perimeter edge, the side wall extending vertically, the side wall surrounding the flat surface and forming an internal space for the baby to rest therein; and an environmental sensor connected to the side wall, the environmental sensor configured to sense environmental parameters such as noise and temperature.

In some aspects, the techniques described herein relate to a system, further including: a processor connected to the sensors, the processor configured to generate sleep recommendations and alerts based on the sensor data; and a graphical user interface to display the sleep recommendations and alert to a care giver of the baby.

In some aspects, the techniques described herein relate to a system, wherein the graphical user interface is configured to receive input from the care giver.

In some aspects, the techniques described herein relate to a system, wherein the system is a bassinet, crib or incubator.

In some aspects, the techniques described herein relate to a computer-implemented method of training a neural network to provide sleep recommendations, information and alerts of an infant, the method including: collecting infant data including physiological parameters, life events and surrounding environment factors; collecting standard data including one or more of health data or educational data; integrating the infant data and standard data into a local dataset; applying one or more transformations to the local dataset to create a preprocessed dataset, the one or more transformation including handling missing values, normalization, standardization, or noise reduction; applying signal processing to the preprocessed dataset to generate a local training set including physiological signals, physiological trends, health or sleep-related parameters; training the neural network using the local training set to recognize local historical health and sleep dataset of the infant; receiving, at a federated learning module in a server, a plurality of historical datasets including the local historical health and sleep dataset and a plurality of other historical datasets corresponding to a plurality of other infants; aggregating the plurality of historical datasets into a federated dataset; training the federated learning module on the federated dataset to identify global patterns and trends and develop infant sleep insights; providing the infant health and sleep insights to the neural network; and using the infant health and sleep insights and local historical data to provide infant health sleep recommendations, via a graphical user interface, to the local infant.

In some aspects, the techniques described herein relate to a method, wherein collecting infant further includes: using a local infant sleep system including integrated sensors to collect physiological data of the infant and environmental data of the infant's sleeping environment.

In some aspects, the techniques described herein relate to a method, wherein receiving, at a federated learning module in a server, a plurality of historical datasets further includes: using a plurality of other infant sleep systems including integrated sensors to collect physiological data of the plurality of other infants and environmental data of the plurality of other infant's sleeping environments.

In some aspects, the techniques described herein relate to a method, wherein collecting standard data further includes: receiving data from computer applications related to infant care.

In some aspects, the techniques described herein relate to a computer-implemented method of training a neural network to recognize for infant sleep care parameters, the method including: collecting infant data while the infant is resting in a bassinet, crib or incubator, the bassinet, crib or incubator including a sensor configured to sense the infant from beneath the infant, the sensor configured to sense physiological parameters or the infant; storing standard data including infant health or infant development parameters; integrating the infant data and the standard data into a local dataset; applying one or more transformations to the local dataset to create a preprocessed dataset, the one or more transformation including handling missing values, normalization, standardization, or noise reduction; applying signal processing to the preprocessed dataset to generate a local training set including physiological signals, physiological trends, health or sleep-related parameters; and training the neural network using the local training set to recognize local historical health and sleep dataset of the local infant.

### BRIEF DESCRIPTION OF THE DRAWINGS

The foregoing summary, as well as the detailed description of the preferred embodiments of the present invention, will be better understood when read in conjunction with the appended drawings. For the purpose of illustrating the invention, there is shown in the drawings, which are diagrammatic, embodiments that are presently preferred. It should be understood, however, that the present invention is not limited to the precise arrangements and instrumentalities shown. In the drawings:

FIG. 1 depicts a front view of a sleep system according to this disclosure;

FIG. 2A is prospective of the sleep system of FIG. 1 including torso sensors according to this disclosure;

FIG. 2B is a side view of the sleep system depicted in FIG. 2A;

FIG. 3A is a prospective view of the sleep system of FIGS. 1, 2A, and 2B with the upper surface removed;

FIG. 3B is a prospective view of the sleep system of FIG. 3A with the upper, flat surface and sensor in place;

FIG. 3C is a top view of the system of FIGS. 2A and 2B;

FIG. 4A is a prospective view of the system of FIGS. 2A and 2B showing the environmental sensor according to this disclosure;

FIG. 4B is an exploded view of section A of FIG. 4A;

FIGS. 5A and 5B depict side views, according to this disclosure, of the system of FIGS. 1, 2A and 2B including the processor within the support;

FIG. 6 is a schematic of a bassinet system according to this disclosure;

FIG. 7 is a flow diagram of a method of training the neural network within the processor of FIGS. 5A and 5B; and

FIG. 8 depicts federated learning among multiple smart infant sleeping systems.

### DETAILED DESCRIPTION OF THE DISCLOSURE

Certain terminology is used in the following description for convenience only and is not limiting. As used herein, the words “connected” or “coupled” are each intended to include integrally formed members, direct connections between two distinct members without any other members interposed therebetween and indirect connections between members in which one or more other members are interposed therebetween. The terminology includes the words specifically mentioned above, derivatives thereof, and words of similar import.

An artificial intelligence powered smart infant sleep system, according to this disclosure, may provide a comprehensive solution for managing infant care routines while alleviating stress on caregivers. The system integrates multi-source data collection, multiple sensor technologies, machine learning models, and a user-friendly interface into a single, holistic system.

The infant sleep system 100 is depicted, for example, in the form of a smart bassinet 5 which may include bassinet insert with lateral sensory guard 18, 19 or as described as “bumpers” in U.S. Pat. Nos. 11,357,336 and 11,825,961 both entitled, “Newborn Sleep Insert for Bassinette and Crib”. These two publications mentioned herein are incorporated by reference to disclose and describe the methods and/or materials in connection with which the publications are cited.

Infant sleep system 100 may also include other forms to accommodate the age and size of an infant or baby that may rest therein. For example, system 100 may be in the form of an incubator to accommodate a pre-mature infant or a crib for an infant older than 6-8 weeks. Further, system 100 may or may not include the lateral sensory guards 18, 19. The smart bassinet 5 may include an array of sensors 20, 21, 24 50, 55 positioned in various locations such as the interior and exterior walls as well as and include lateral sensor guards 18, 19 which may contact portions of the infant such as the torso or arms of the infant, etc. In these positions, the sensors monitor the infant's physiological parameters, such as heart rate, body temperature, movement, and breathing patterns. Moreover, environmental factors that might influence the infant's comfort and sleep, like room temperature, noise levels, and light intensity, are also tracked continuously. These data points, coupled with information inputted manually by parents and integrated from other partner apps are continuously collected and transmitted to an onboard processor for analysis. It is noted that system 100 may include at least one lateral sensory guard 18, 19 or both lateral sensory guards 18, 19.

System 100 includes integration of multiple sensors to monitor a wide range of physiological and environmental parameters. By leveraging federated learning, the system processes this data to provide personalized, evidence-based recommendations aimed at improving infant sleep and health outcomes. The inclusion of an open API ecosystem further enhances the system's capabilities by allowing third-party developers to create innovative applications that utilize the insights generated by the bassinet's sensors.

Moreover, the data crowd-sourcing approach not only improves the system's 100 recommendations by learning from a large cohort of infants but also creates invaluable datasets for research and medical institutions. This vast data pool can be used to advance our understanding of infant health and develop new strategies for preventing conditions such as SIDS. The collaboration with research and medical institutions ensures that the system remains at the forefront of scientific and medical advancements, ultimately contributing to the well-being of infants and reducing parental stress and anxiety.

In essence, this invention provides a holistic solution that combines cutting-edge technology with a collaborative approach to revolutionize infant care, ensuring safer and more effective sleep management for infants and peace of mind for parents.

This integration of features provides a dynamic, personalized system capable of learning and adapting to the unique needs of each infant and their caregivers. By leveraging advanced technology, machine learning, and a vast dataset, the AI-Powered Smart Bassinet offers a scientifically backed, holistic approach to managing infant care routines. It effectively reduces stress for caregivers while promoting the health, safety, and comfort of infants.

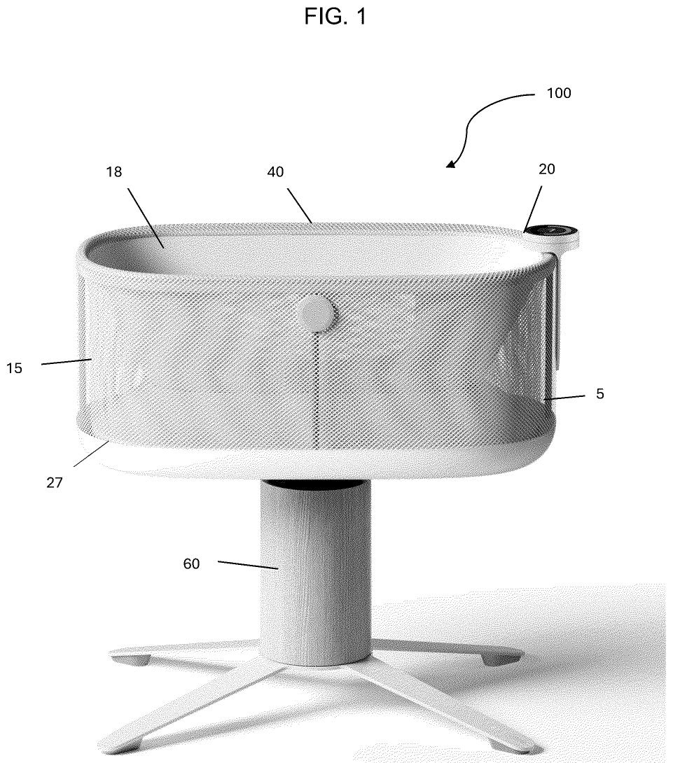

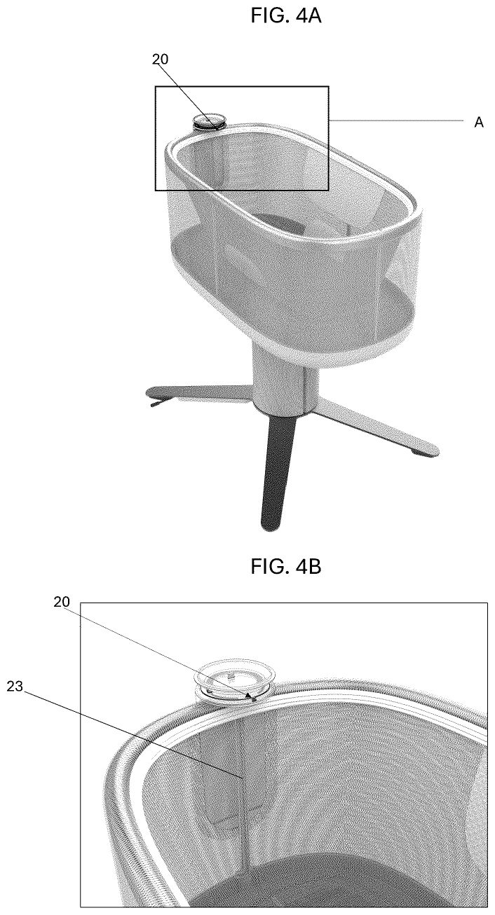

Referring now to the drawings in detail, wherein like numbers are used to indicate like elements throughout, FIGS. 1 and 2A-5B depict a smart bassinet or infant sleep system 100 including a bassinet 5 including environmental sensor 20, physiological sensor 50, lateral sensory guards 18, 19 and torso or lateral sensors 21, 24, graphical user interface (“GUI”) 70, processor 80 and global federated learning module 200 in remote server 300.

Bassinet 5 includes sidewall 15, sleep surface or mattress 30, upper or flat surface 26, and support 60. Sidewall 15 extends between top rail 40 and upper surface 26, and sidewall 15 is connected to upper surface 26 at the perimeter edge 27 of upper surface 26. Further, sidewall 15 may be formed of mesh and may entirely surround upper surface 26.

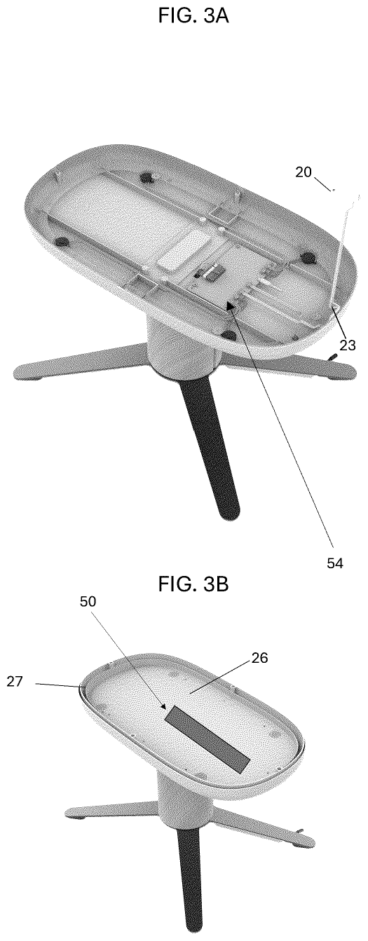

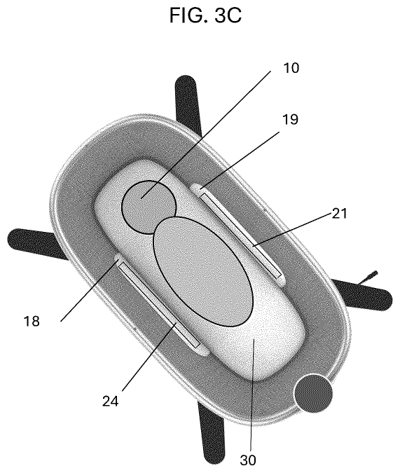

Surface 26 extends horizontally and provides a flat surface to support a resting infant or baby 10. Preferably, mattress 30 rests on upper surface 26 and physiological sensor 50 is therebetween. As such, mattress 30 may upper surface 26 and sensor 50. Also, sensor 50 may be held in place on upper surface 26 with an adhesive or clips, etc. As shown in FIG. 3B-3C, it is preferable that sensor 50 is centrally positioned on upper surface 26 such that sensor 50 is configured to be beneath a baby 10 resting on mattress 30.

Physiologic sensor 50 is positioned between the bottom surface of the mattress 30 and upper surface 26. Sensor 50 may be an electromagnetic film sensor such as an EMFIT or photoplethysmogram, Respiratory Inductive Plethysmography (RIP) Sensor, Piezoelectric Respiratory Sensor, or Accelerometer, and include a module 60 for both powering sensor 50 and connection of sensor 50 to processor 80. Sensor 50 may monitor one or more physiological parameters of the infant including heart rate, body temperature, movement, and breathing patterns. For each of the parameters monitored, sensor 50 communicates physiological data 52 to processor 80.

In addition to sensor 50 located under the mattress 30, system 100 also includes sensors 21, 24 positioned on the lower lateral sides of the bassinet 5 and contained in or on lateral sensory guards 19, 18, respectively. As lateral sensory guards 19, 18 extend vertically above mattress 30, sensors 21, 24 are configured to sense the torso or side of baby 10. Lateral sensors 21, 24 may provide additional readings from the lateral torso of the infant, capturing a more comprehensive set of physiological data. The integration of sensors 21, 24 allows for enhanced monitoring of the infant's breathing patterns, movements, and other vital signs, contributing to a more accurate and holistic understanding of the infant's well-being.

Environmental sensor 20 may be connected to top rail 40. This position allows sensor 20 to measure one or multiple environmental factors including room temperature, noise levels, and light intensity, etc. surrounding bassinet 5. Sensor 20 generates environmental data 22 corresponding to each measured factor and communicates data 22 to processor 80. Alternatively, sensor 20 may be connected to an outer or external surface of sidewall 15 or top rail 40 or spaced apart from the basinet 5 and communicate wirelessly with processor 80. When sensor 20 is connected to bassinet 5, sensor 20 may communicate with processor 80 via a wired 23 or wireless connection. Although one environmental sensor 20 is discussed herein, multiple sensors may be utilized and positioned exterior to bassinet 5 and/or interior to bassinet 5.

Regarding noise or audio sensing, sensor 20 monitors environmental sounds and verbal activities of the infant 10. For example, sensor 20 may recognize and record crying and which may include time of crying, length of crying, and volume, pattern of cry. Sensor 20 generates audio data corresponding to environmental sounds or verbal activities of the infant 10 such as cry data 57 and communicates data 57 to processor 80.

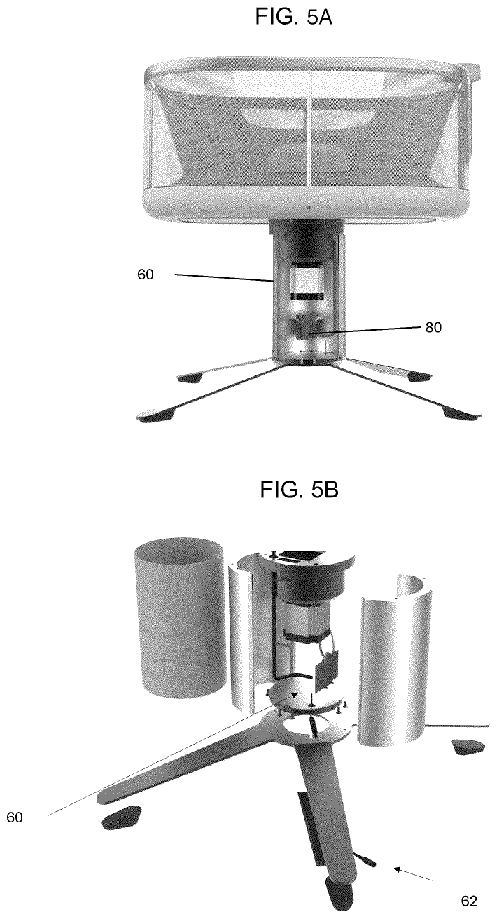

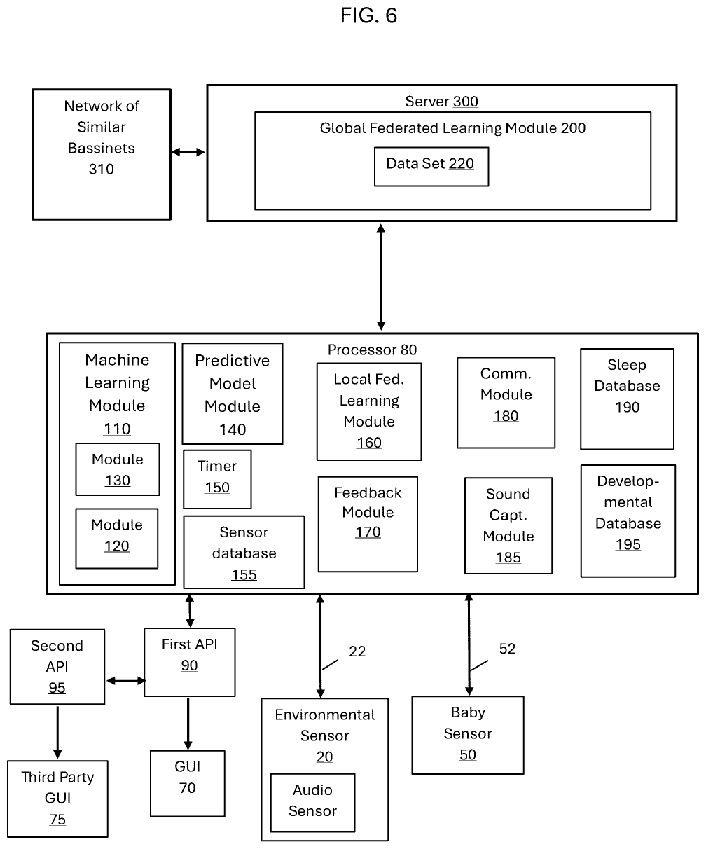

Processor 80 is in communication with sensors 20, 21, 24, 50 and may be embedded within bassinet 5. For example, processor 80 may be within support 60 as shown in FIGS. 5A and 5B. Internally, processor 80, as shown in FIG. 6, may include machine learning module 110, at least one convolutional neural network (“CNN”) module 120, at least one recurrent neural network module 130 (“RNN”), predictive model module 140, timer 150, sensor database 155, local federated learning module 160, caregiver feedback module 170, communication module 180, sleep database 190, and developmental database 195. Modules 110, 120, 130, 140 may receive and process data 22, 52, 57. Machine learning module 110 applies advanced machine learning algorithms and predictive modeling technologies to data 22, 52, 57. Further, module 110 includes CNN module 120 and RNN module 130. CNN module 120 extracts patterns from data 22, 52, 57 and identifies correlations between different variables (e.g. physiological, environmental, health and sleep related). RNN module 130 uses data 52 to understand the baby's sleep behavior over time with reference to timer 150. Predictive model module 140 employs historical sleep pattern data and caregiver feedback to anticipate potential sleep disruptions.

Sensor database 155 may store any data received from sensors 20, 21, 24, 50.

Parent feedback module 170 receives feedback, via a bi-directional GUI 70, from infant caregivers (e.g. parent(s), guardian, and/or nurse) as whether or not recommendations made by processor 80 are effective in assisting infant/baby 10 to improve sleep habits. Timer 150 may include a chronological timer, or other types of timers needed to monitor the infant 10 and/or environment. Module 170 is also configured to display on the GUI any recommendations generated by modules 110, 140, 160, and 185 for the caregivers to improve the infant sleep habits. Communications module 180 allows for wired and wireless (e.g. UHF radio and Wi-Fi) communications between bassinet 5 and processor 80.

Cry module 185 receives audio data 57 from environmental sensor 20. Module 185 includes advanced cry recognition capabilities, utilizing audio data 57 and machine learning algorithms to distinguish between different types of crying and alert caregivers to potential needs or issues via GUI 70.

Sleep database 190 may include the historical sleep pattern data and care giver feedback. Developmental database 195 includes infant developmental milestones for the various stages of an infant's life. Each database 190, 195 may be referenced by modules 110, 120, 130, 140 for use in system 100 algorithms, thereby offering age-appropriate recommendations and insights.

Local federated learning module 160 prepares and sends data to a global federated learning module 200 in a remote server 300. Module 200 includes an extensive range of infant health, sleep patterns and routines, enhancing the system's understanding of various sleep behaviors and conditions using a large dataset 220 of anonymized infant sleep, environmental, and health data from a network of similar smart bassinets. This dataset 220 provides an extensive range of infant sleep patterns, routines, environmental, and health data, enhancing the system's understanding of various sleep behaviors. By cross-referencing this dataset 220, system 100, via module 160, identifies common patterns and best practices, effectively providing caregivers with an evidence-based approach to care routines. Processor 80 may then make recommendations on GUI 70 to infant care givers to improve the sleep patterns, overall well-being and development of baby 10.

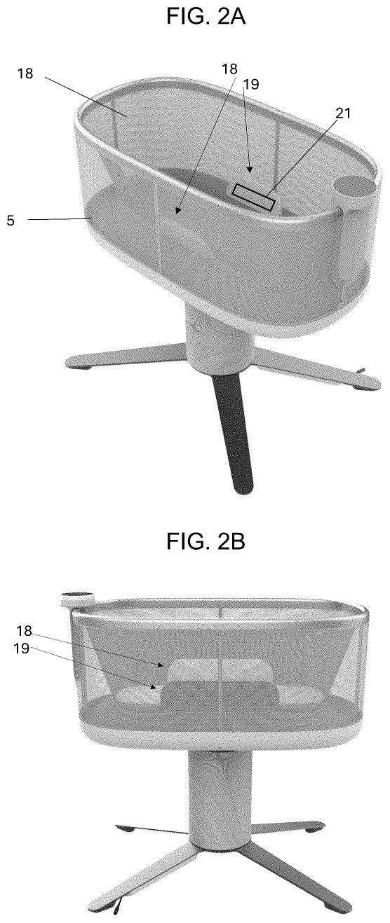

GUI 70 may be attached to base 60, as shown in FIG. 2 and/or GUI 70 may be incorporated into a stand-alone, portable, or remote device such as smart phone, laptop, desktop and/or tablet, etc. GUI 70 receives and displays personalized recommendations, corresponding infant 10 and generated by processor 80. Additionally, GUI 70 receives caregiver feedback, enabling the system to continuously refine its understanding and adaptation to the infant's sleep patterns and routines. The intuitive design of the interface, employing user-friendly graphics and straightforward language, makes the system's recommendations easily understood and implemented by caregivers.

Additionally, system 100 may include a dual-Application Programming Interface (“API”) architecture to enhance functionality and data security. The first API 90 is designed for on-device data processing, where machine learning algorithms directly interact with data stored in sensor database 155 to generate real-time insights and recommendations without transmitting raw data externally. This API supports local computations and adaptations, ensuring swift response times and reducing dependency on external data processing.

The second API 95 manages the secure transmission of processed data to external applications and third-party developers. It is crafted to ensure that data, while being accessible for further innovation and application development, remains encrypted and compliant with privacy standards. This second API facilitates the expansion of the ecosystem, allowing third-party developers to create bespoke applications that utilize the insights generated by the bassinet's sensors, thus contributing to an integrated care solution platform.

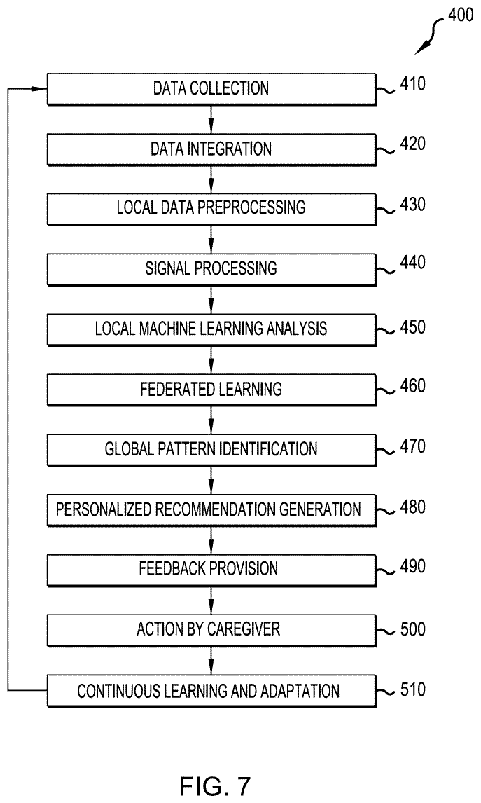

FIG. 7 is a schematic of a method 400, according to this disclosure, of promoting infant sleep and development using smart bassinet infant sleep system 100. Additionally, method 400 is a computer-implemented method of training a neural network to provide sleep recommendations of a local infant.

Step 410 includes data collection. The embedded sensors or remote sensors 20, 50 in bassinet 5 continuously collect data 22, 52 relating to the infant's physiological parameters such as body temperature, movement, crying patterns, breathing patterns, and heart rate. Additional parameters such as environmental factors (room temperature, noise, light levels) are also recorded. Moreover, caregivers can manually input data such as feeding times, diaper changes, or notable behaviors. Standard data such as data from other partner apps, such as nutrition or healthcare apps, and educational health or developmental data may also be integrated. Data 22, 52 may be collected with sensors 20, 50, 21, 24.

Next, step 420, includes data integration. All data collected in step 410 such as data 22, 52 from sensors 20, 50, data from parental inputs via GUI 70, and partner apps are consolidated into a comprehensive data set ready for further processing and analysis.

Further, in step 430, local data preprocessing is performed. The integrated data undergoes cleaning, organizing, and structuring into a format suitable for further analysis. This includes handling missing values, normalization, standardization, and noise reduction.

Next, in step 440, signal processing is performed. Key features from physiological signals are extracted, patterns and trends are analyzed, signal enhancement techniques are applied, and sleep-related parameters are identified.

Next, in step 450, local machine learning analysis is performed. The processed data is analyzed using algorithms in the machine learning module 110, CNN module 120 and RNN module 130 to understand the infant's unique behaviors, needs, and routines. Leveraging supervised and deep learning algorithms (e.g., convolutional neural networks, recurrent neural networks), the system is trained on historical sleep data.

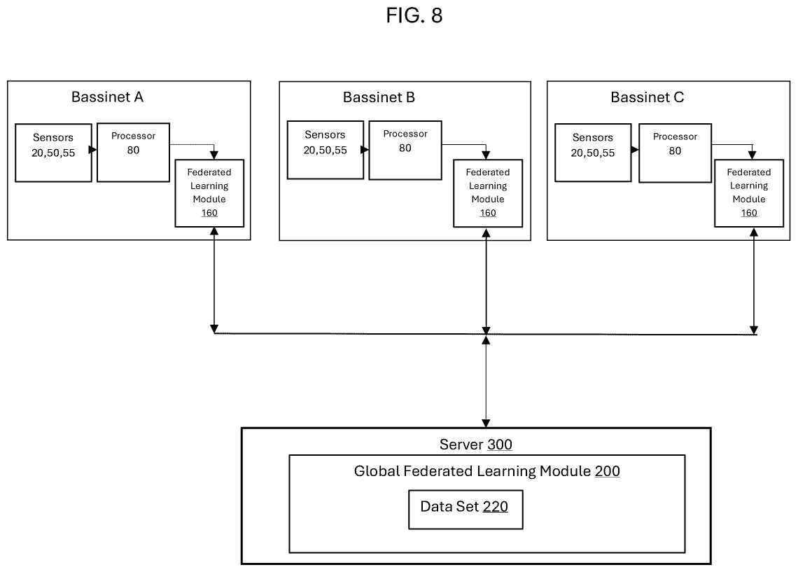

In step 460, federated learning occurs. System 100 shares insights from the local machine learning analysis with a larger network 310 of similar smart bassinets. The data shared at this stage is anonymized and aggregated to ensure privacy and shared via global federated learning module 200. It is noted that system 100 may be part of network 310. FIG. 8 is a schematic of federated learning among a plurality of bassinet systems (eg. Bassinet A, B, C) similar or the same as system 100.

In step 470 dataset 220 is developed and continually refined in global federated learning module 200. The aggregated data from multiple smart bassinets is

Analyzed to identify global patterns and trends. This step is essential to identify commonalities and differences and analyze the overall dataset 220 to identify broader trends.

Next, step 480 includes personalized recommendation Generation. Based on both the local machine learning analysis of step 450 and the insights from the federated learning of steps 460, 470, the system generates personalized, age-appropriate recommendations. This includes suggestions for optimal feeding times, sleep times, and room temperatures, and potential health alerts.

Step 490 includes feedback provision. System may provide real-time feedback and predictions to the caregivers via an intuitive GUI 70. This could be a mobile app or an integrated display in the smart bassinet.

Step 500 includes action(s) by caregiver(s). Based on the recommendations and feedback provided in step 490, a caregiver may adjust their infant care practices accordingly. Further, GUI 70 allows the caregiver to communicate to system 100 if the recommendations are successful. Thereby, allowing system 100 to continually learn.

Step 510 includes continuous learning and adaptation. System 100 continuously learns and adapts from the new data generated by the infant's behavior and the caregiver's actions. This ongoing cycle of data collection, analysis, and recommendation generation ensures that the system stays updated and the recommendations remain relevant as the infant grows and develops.

This flowchart of method 400 shows the cyclical nature of the system's operations and the constant learning and adaptation that goes into providing personalized and effective infant care recommendations. The multi-source data collection, combined with local and federated machine learning, creates a uniquely comprehensive approach to infant care.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as generally defined in the appended claims.

1. A sleep system for a baby comprising:
a flat surface configured to support the baby;
a physiological sensor positioned beneath the flat surface;
a mattress over the flat surface and the physiological sensor such that the physiological sensor is configured to sense from beneath the baby; and
at least one lateral sensor connected to the flat surface and configured to sense the baby from a side of the baby,
wherein the at least one lateral sensor and the physiological sensor are configured to sense at least one at least one physiological parameter of the baby and generate corresponding sensor data.

2. The system of claim 1, wherein the least one lateral sensor extends above the mattress.

3. The system of claim 1, further comprising:
a perimeter edge surrounding the flat surface;
at least one lateral sensory guard extending vertically from the flat surface, the lateral sensory guard between the perimeter edge and a central portion of the mattress; and
the at least one lateral sensor on the lateral sensory guard.

4. The system of claim 1, further comprising:
a perimeter edge surrounding the flat surface;
a side wall connected to the perimeter edge, the side wall extending vertically, the side wall surrounding the flat surface and forming an internal space for the baby to rest therein; and
an environmental sensor connected to the side wall, the environmental sensor configured to sense environmental parameters such as noise and temperature.

5. The system of claim 1, further comprising:
a processor connected to the sensors, the processor configured to generate sleep recommendations and alerts based on the sensor data; and
a graphical user interface to display the sleep recommendations and alert to a care giver of the baby.

6. The system of claim 5, wherein the graphical user interface is configured to receive input from the care giver.

7. The system of claim 1, wherein the system is a bassinet, crib or incubator.

8. A computer-implemented method of training a neural network to provide sleep recommendations, information and alerts of an infant, the method comprising:
collecting infant data including physiological parameters, life events and surrounding environment factors;
collecting standard data including one or more of health data or educational data;
integrating the infant data and standard data into a local dataset;
applying one or more transformations to the local dataset to create a preprocessed dataset, the one or more transformation including handling missing values, normalization, standardization, or noise reduction;
applying signal processing to the preprocessed dataset to generate a local training set including physiological signals, physiological trends, health or sleep-related parameters;
training the neural network using the local training set to recognize local historical health and sleep dataset of the infant;
receiving, at a federated learning module in a server, a plurality of historical datasets including the local historical health and sleep dataset and a plurality of other historical datasets corresponding to a plurality of other infants;
aggregating the plurality of historical datasets into a federated dataset;
training the federated learning module on the federated dataset to identify global patterns and trends and develop infant sleep insights;
providing the infant health and sleep insights to the neural network; and
using the infant health and sleep insights and local historical data to provide infant health sleep recommendations, via a graphical user interface, to the local infant.

9. The method of claim 8, wherein collecting infant further comprises:
using a local infant sleep system including integrated sensors to collect physiological data of the infant and environmental data of the infant's sleeping environment.

10. The method of claim 8, wherein receiving, at a federated learning module in a server, a plurality of historical datasets further comprises:
using a plurality of other infant sleep systems including integrated sensors to collect physiological data of the plurality of other infants and environmental data of the plurality of other infant's sleeping environments.

11. The method of claim of claim 8, wherein collecting standard data further comprises:
receiving data from computer applications related to infant care.

12. A computer-implemented method of training a neural network to recognize for infant sleep care parameters, the method comprising:
collecting infant data while the infant is resting in a bassinet, crib or incubator, the bassinet, crib or incubator including a sensor configured to sense the infant from beneath the infant, the sensor configured to sense physiological parameters or the infant;
storing standard data including infant health or infant development parameters;
integrating the infant data and the standard data into a local dataset;
applying one or more transformations to the local dataset to create a preprocessed dataset, the one or more transformation including handling missing values, normalization, standardization, or noise reduction;
applying signal processing to the preprocessed dataset to generate a local training set including physiological signals, physiological trends, health or sleep-related parameters; and
training the neural network using the local training set to recognize local historical health and sleep dataset of the local infant.
